(19) United States
(12) Patent Application Publication
(10) Pub. No.: US 2023/0225365 A1
(43) Pub. Date: Jul. 20, 2023

(54) METHODS OF PRODUCING BUTYRATE PRODUCTS

(71) Applicant: SUPERBREWED FOOD, INC., New Castle, DE (US)

(72) Inventors: Aharon Eyal, Jerusalem (IL); Shawn William Jones, Bear, DE (US); Bryan P. Tracy, Wilmington, DE (US)

(21) Appl. No.: 18/010,056

(22) PCT Filed: Jun. 15, 2021
(86) PCT No.: PCT/IB2021/055253

§ 371 Date: Dec. 13, 2022

Related U.S. Application Data
(60) Provisional application No. 63/039,566, filed on Jun. 16, 2020.

(51) Int. Cl. A23K 10/12; C12P 7/52; A23K 20/105; A23K 10/14

(52) U.S. Cl. CPC A23K 10/12; C12P 7/52; A23K 20/105; A23K 10/14

(57) ABSTRACT

Provided is a method for the manufacture of a butyrate product by fermentation of a carbon source-containing fermentation feedstock with a Clostridia class bacterium which natively produces butyric acid while maintaining a pH of about 5 to 7, wherein ammonia is used for said maintaining pH and whereby a fermentation broth comprising ammonium butyrate, a biomass, and optionally a fermentation by-product is formed; separating said biomass from said fermentation broth to form separated biomass and a clarified fermentation broth, wherein said clarified fermentation broth comprises said ammonium butyrate and optionally said fermentation by-product; and adding to said clarified broth an additive selected from the group consisting of a mineral acid, a mineral base, a soluble calcium salt and combinations thereof, whereby said butyrate product is formed in said clarified broth. Further provided are feed ingredients, animal feeds and anti-icing agents comprising the composition, and uses thereof.

### CROSS-REFERENCE TO RELATED APPLICATION

The present application gains priority from U.S. Provisional Application No. 63/039,566 filed Jun. 16, 2020, which is incorporated by reference as if fully set-forth herein.

### FIELD OF THE INVENTION

The present invention relates to methods of producing butyrate products and more specifically to methods comprising fermentation of a carbon source-containing feedstock with a Clostridia class bacterium which natively produces butyric acid, and further processing of the fermentation product to produce the butyrate product.

### BACKGROUND

There is an ongoing need for improved butyrate products, such as feed ingredients that better achieve improvements, such as increased feed intake, improved feed conversion rate, faster daily weight gain, greater ileum surface area and consistent results between sexes when fed to an animal.

### SUMMARY OF THE INVENTION

According to an aspect of some embodiments of the present invention, provided is a method for manufacturing a butyrate product, the method comprising:

(i) providing a carbon-source-comprising fermentation feedstock;
(ii) fermenting said feedstock with a Clostridia class bacterium which natively produces butyric acid, while maintaining a pH of about 5 to 7, wherein ammonia is used for said maintaining pH, whereby a fermentation broth comprising ammonium butyrate, a biomass, and optionally a fermentation by-product is formed;
(iii) separating said biomass from said fermentation broth to form separated biomass and a clarified fermentation broth, wherein said clarified fermentation broth comprises said ammonium butyrate and optionally said fermentation byproduct; and
(iv) adding to said clarified broth an additive selected from the group consisting of a mineral acid, a mineral base, a soluble calcium salt and combinations thereof, whereby said butyrate product is formed in said clarified broth.

According to a further aspect of some embodiments of the present invention, provided is an animal feed ingredient comprising the butyrate product as disclosed herein.

According to a further aspect of some embodiments of the present invention, provided is an anti-icing agent comprising the butyrate product as disclosed herein.

According to a further aspect of some embodiments of the present invention, provided is a method for treating an animal comprising feeding said animal with a feed comprising the feed ingredient as disclosed herein.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a line graph showing metabolite and cell production and glucose consumed as a function of fermentation time for Clostridium tyrobutyricum batch fermentation with glucose as the feedstock

### DETAILED DESCRIPTION OF THE INVENTION

The present invention relates to a method for manufacturing a butyrate product comprising fermentation of a carbon source-containing feedstock with a Clostridia class bacterium which natively produces butyric acid, and further processing of the fermentation product to produce the butyrate product.

The further processing results in an improved butyrate product as compared to that obtained by the fermentation process alone.

The improved butyrate product may be a purified butyrate product and/or a butyrate product in solid form (such as a butyrate salt, which may be, for example, a salt of calcium or sodium) which are advantageous for use in a feed ingredient. Butyrate salts of calcium and sodium have been found to be preferable to butyrate salts of ammonium as feed ingredients.

The improved butyrate product may have a low phosphorus content, wherein the method comprises removal of phosphorus from the clarified fermentation broth. Such low-phosphorus products are advantageous for use as an anti-icing agent. Furthermore, it has been found that potassium salts of acetate or butyrate have better anti-icing effects that those obtained by a corresponding ammonium salt.

The improved butyrate product may be obtained by a lower cost process as compared to processes known in the art for obtaining such products. The reduced cost may be due to reusing ammonia liberated upon conversion of ammonium butyrate into the butyrate product and/or from the use of less refined carbon sources that include impurities such as phosphorus anions, as well as due to the use of processing coproducts such as calcium phosphate as a feed ingredient and as a fertilizer.

The further processing step may comprise, for example, separating the butyrate product from co-products of fermentation, such as by distilling the butyrate product from the fermentation broth, distilling or vaporizing a co-product of fermentation from the fermentation broth, or forming an insoluble salt of a phosphorus anion, such as phytate or phosphate, which are found as impurities in prior art methods of fermentation and removing the insoluble salt from the fermentation broth.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “butyrate product” refers to a compound containing or derived from butyrate such as a butyrate salt, butyric acid, butyrate ester and the like.

As used herein, the term “feed ingredient” refers to a component, part, constituent or any combination/mixture to an animal food.

As used herein, the term “fermentation feedstock” refers to a component or components added to a fermentation.

As used herein, the term “animal feed” refers to a product intended for consumption by a non-human animal, such as livestock, poultry, companion animal, or the like.

As used herein, the term “anti-icing agent” refers to a material which can remove ice from a surface and/or prevent or reduce formation of ice on a surface and encompasses deicing agents.

As used herein, the term “protected” with regard to butyrate refers to a butyrate molecule that is provided in a bound or reversibly reacted form in order to enable the molecule to pass through the upper gastrointestinal tract.

As used herein, the term “treating” includes ameliorating, mitigating, and reducing the instances of a disease or condition, or the symptoms of a disease or condition.

As used herein, both terms “vaporizing” and “distilling” mean transfer from a liquid phase into a vapor phase and are used interchangeably.

As used herein, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed. As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

According to an aspect of some embodiments of the present invention, provided is a method for manufacturing a butyrate product, the method comprising (i) providing a carbon-source-comprising fermentation feedstock; (ii) fermenting said feedstock with a Clostridia class bacterium which natively produces butyric acid, while maintaining a pH of about 5 to 7, wherein ammonia or ammonium hydroxide is used for said maintaining pH and whereby a fermentation broth comprising ammonium butyrate, a biomass, and optionally a fermentation by-product is formed (iii) separating said biomass from said fermentation broth to form separated biomass and a clarified fermentation broth, wherein said clarified fermentation broth comprises said ammonium butyrate and optionally said fermentation byproduct; and (iv) adding to said clarified broth an additive selected from the group consisting of a mineral acid, a mineral base, a soluble calcium salt and combinations thereof, whereby said butyrate product is formed in said clarified broth.

According to an embodiment, said separating said biomass comprises at least one of filtering and centrifuging.

According to an embodiment, the carbon source is selected from the group consisting of starch, dextrose, maltodextrin, glucose, liquefied corn mash, fructose, xylose, glycerol, sucrose, hemicellulose, cellulose and combinations thereof.

According to an embodiment, said additive comprises a mineral acid, said butyrate product formed in said clarified broth is butyric acid in free acid form, and the method further comprises separating said butyric acid in free acid form from said clarified broth by a method selected from the group consisting of distilling and liquid-liquid extraction of said butyric acid.

According to an embodiment, said additive comprises a mineral acid, butyric acid in free acid form and an ammonium salt of said mineral acid are formed in said clarified broth, and the method further comprises providing an alkanol, reacting said butyric acid in free acid form with said alkanol to form a butyrate ester, and separating said butyrate ester from said clarified broth by a method selected from the group consisting of distilling and liquid-liquid extraction of said butyric ester.

According to an embodiment, said alkanol is selected from the group consisting of methanol, ethanol, propanol, butanol and combination thereof.

According to an embodiment, the pH of said clarified broth is adjusted to 2-5 prior to or simultaneously with said reacting.

According to an embodiment, said mineral acid is selected from the group consisting of sulfuric acid, phosphoric acid and nitric acid and combinations thereof.

According to an embodiment, said additive comprises a mineral base, ammonia is liberated in said clarified broth, said butyrate product formed in said clarified broth is a butyric salt of said mineral base, and the method further comprises separating said butyrate product from said liberated ammonia by vaporizing said liberated ammonia. According to an embodiment, said vaporizing is conducted, at least partially, at atmospheric pressure or at a pressure lower than atmospheric (e.g. 0.2-1 atmosphere) and at a temperature below water boiling point at that pressure. According to an embodiment, said vaporizing comprises steam distillation.

According to an embodiment, said mineral base is selected from the group consisting of oxides, hydroxides, carbonates and bicarbonates of sodium, potassium, calcium and magnesium and combinations thereof.

According to an embodiment, at least one selected from the group consisting of said clarified fermentation broth, said carbon source and said carbon-source containing fermentation feedstock further comprises a phosphorous anion selected from the group consisting of phosphate, phytate and combinations thereof and said additive comprises a soluble calcium salt, wherein a water-insoluble calcium salt of said phosphorous anion is formed, and the method further comprises separating said butyrate product from said insoluble salt. According to some such embodiments, separating said butyrate product from said insoluble salt comprises removing said insoluble salt, e.g. by filtration or centrifugation of said insoluble salt. According to an embodiment, the pH of the clarified broth is adjusted to 7.5 to 9.5 prior to or simultaneously with adding said additive, but prior to said removing.

According to an embodiment, said carbon-source and/or said carbon-source containing fermentation feedstock comprises phytate, the method further comprising contacting said carbon-source and/or said carbon-source containing fermentation feedstock with a phytase enzyme, whereby phytase is hydrolyzed to form phosphate, wherein said contacting with phytase enzyme is conducted prior to or simultaneously with said fermenting.

According to an embodiment, said soluble calcium salt has water solubility greater than 1% by weight, such as at least 1%, at least 2%, at least 5%, at least 10%, at least 20%, at least 30%, at least 40%, at least 50%, at least 60%, or even at least 70% by weight.

According to an embodiment, said soluble calcium salt is selected from the group consisting of calcium acetate, calcium propionate, calcium butyrate, calcium chloride and combination thereof.

According to an embodiment, said fermentation broth further comprises ammonium acetate.

According to an embodiment, said butyrate product is a feed ingredient.

According to an embodiment, said butyrate product is an anti-icing agent.

According to an embodiment, a phosphorous content of said anti-icing agent is less than 0.1% by weight, less than 0.08%, less than 0.06%, less than 0.04%, less than 0.02% or less than 0.01%.

According to an embodiment, said additive comprises a mineral base, and a butyrate salt of said mineral base is formed in said clarified broth, and the method further comprises separating said butyrate salt of said mineral base from said clarified broth by at least one method selected from the group consisting of salt crystallization and electrodialysis.

According to an embodiment, said additive comprises a mineral acid, and an ammonium salt of said mineral acid is formed in said clarified broth, and the method further comprises crystallizing said ammonium salt of said mineral acid from said clarified broth.

According to an embodiment, said ammonium salt of said mineral acid is selected from the group consisting of ammonium sulfate, ammonium phosphate, ammonium nitrate and a combination thereof. According to an embodiment, there is provided a fertilizer comprising the ammonium salt as disclosed herein.

According to an embodiment, the method further comprises reacting said ammonium salt of said mineral acid with a calcium base, whereby ammonia is liberated and a precipitate is formed and wherein said precipitate comprises calcium sulfate, calcium phosphate or a combination thereof. According to some such embodiments, calcium phosphate is formed. According to an embodiment, there is provided an animal feed comprising the calcium phosphate as disclosed herein.

According to another embodiment, there is provided a fertilizer comprising the calcium phosphate as disclosed herein.

According to an embodiment, wherein ammonia is liberated, the method further comprises reusing said liberated ammonia and/or said vaporized ammonia for pH control in said fermentation.

According to an embodiment, the additive comprises a potassium base and potassium butyrate is the butyrate product.

According to an embodiment, the method further comprises protecting at least a fraction of the butyrate in said clarified fermentation broth or in products thereof, such as at least 20%, at least 30%, at least 40%, at least 50%, at least 60%, at least 70%, at least 80% or even at least 90% of the butyrate.

According to an embodiment, the method further comprises at least partially removing water from said fermentation broth and/or from said clarified fermentation broth.

According to some such embodiments, the biomass is first separated from the fermentation broth and the clarified fermentation broth is concentrated to produce a concentrated clarified fermentation broth comprising up to about 60% solutes by weight. According to some such embodiments, the additive is added to the concentrated clarified fermentation broth. According to alternative embodiments, the additive is added to the clarified fermentation broth prior to concentrating. According to further alternative embodiments, the clarified broth is partially concentrated, the additive is then added and the clarified broth is further concentrated.

According to an aspect of some embodiments of the present invention, there is provided an animal feed comprising selected nutrients, a butyrate feed ingredient as disclosed herein or a product thereof and optionally biomass.

According to an embodiment, the animal feed further comprises at least one mineral micronutrient. According to some such embodiments, a mineral micronutrient is a metal ion selected from the group consisting of ions of iron, ions of zinc and combinations thereof. Without wishing to be bound to any one theory, the present Inventors hypothesize that such metal ions form complexes with butyrate (and/or acetate, when present) which increases their bioavailability.

According to an aspect of some embodiments of the present invention, there is provide a method for treating an animal comprising feeding said animal with the feed as disclosed herein.

According to an aspect of some embodiments of the present invention, there is provided the feed as disclosed herein for use in treating an animal.

According to an embodiment, said feed comprises butyrate at a concentration between 10 ppm and 20,000 ppm, such as at least 10 ppm, at least 50 ppm, at least 100 ppm, at least 500 ppm, at least 1,000 ppm, at least 5,000 ppm, at least 10,000 ppm, less than 20,000 ppm, less than 10,000 ppm, less than 5,000 ppm, less than 1,000 ppm, less than 500 ppm, less than 100 ppm or less than 50 ppm.

According to an embodiment, at least 50% by weight of the butyrate in said feed results from said feed ingredient, such as at least 50%, at least 60%, at least 70% or at least 80%.

According to an embodiment, said animal is selected from the group consisting of avian, porcine, bovine, equine, ovine, piscine, caprine, canine, feline and lapine groups of animals.

According to an embodiment, the method provides an improved result compared with feeding same selected nutrients without said feed ingredient.

According to an embodiment, said improved result comprises at least one of increased feed intake, improved feed conversion rate, faster daily weight gain, greater ileum surface area, and consistent results between sexes.

According to an embodiment, said improved result comprises at least one, at least two, at least three, or at least four selected from the group consisting of increased feed intake, improved feed conversion rate, faster daily weight gain, greater ileum surface area, consistent results between sexes and combinations thereof.

According to an embodiment, said method provides an improved result compared with feeding same selected nutrients without said feed ingredient. According to an embodiment, said improved result comprises increased feed intake. According to an embodiment, feed intake increases by at least 0.5%, at least 1%, at least 2%, at least 3%, or at least 4%. According to an embodiment, said improved result comprises improved feed conversion rate (FCR). According to an embodiment, FCR improves by at least 0.1%, at least 0.2%, at least 0.3%, at least 0.4%, at least 0.5%, at least 1%, at least 2.5%, at least 3%, at least 4%, at least 5%, at least 6%, at least 7%, at least 8%, at least 9%, or at least 10%.

According to an embodiment, said improved result comprises faster daily weight gain. According to an embodiment, daily weight gain increases by at least 0.1%, at least 0.2%, at least 0.3%, or at least 0.4%.

### EXAMPLES

### Example 1: Glucose Fermentation

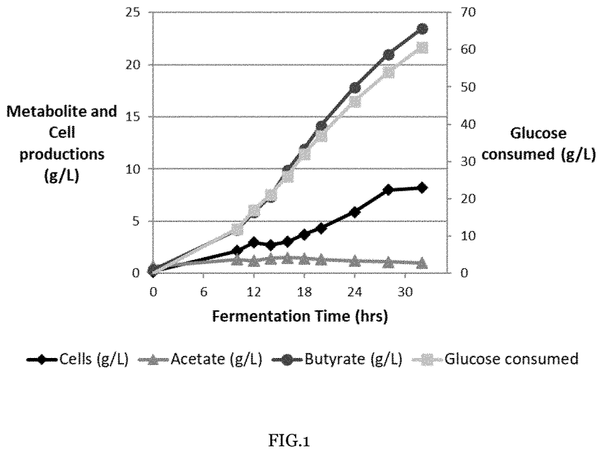

A glucose-based fermentation media, containing 60-160 g/L of glucose, was fermented in a batch fermentation with Clostridium tyrobutyricum. Additional media components include those listed in Tables 1, 2, and 3. Temperature was maintained at 35° C. and pH was controlled at minimum of 5.7 with 8 M NH₄OH. Results of glucose consumption, cell production and metabolite production during 30 hours of fermentation are shown in FIG. 1. Butyrate concentration and cell concentration reached about 60 g/L and 20 g/L, respectively.

TABLE 1
Bulk media components.

| Component | Concentration (g/L) |
|---|---|
| KH2PO4 | 3 |
| K2HPO4 *3H2o | 0.1975 |
| NaCl | 0.6 |
| MgSO4*7H2o | 0.24 |
| MnSO4*H2O | 0.01 |
| FeSO4*7H2O | 0.03 |
| (NH4)2SO4 | 1.5 |
| CaCl2*2H2O | 0.16 |
| Antifoam | 0.1 |

TABLE 2
Trace elements added to media.

| Component | Concentration (mg/L) |
|---|---|
| MnSO4*H2O | 20 |
| Fe(SO4)2(NH4)2*6H2O | 16 |
| CoCl2*6H2O | 4 |
| ZnSO4*7H2O | 0.004 |
| CuCl2*2H2O | 0.4 |
| NiCl2*6H2O | 0.4 |
| Na2MoO4*2H2O | 0.4 |
| Na2SeO4 | 0.4 |
| Na2WO4 | 0.4 |

TABLE 3
Vitamins added to media.

| Component | Concentration (mg/L) |
|---|---|
| Biotin | 0.04 |
| Folic Acid | 0.04 |
| Pyridoxine-HCl | 0.2 |
| Thiamine-HCl | 0.1 |
| Riboflavin | 0.1 |
| Nicotinic acid | 0.1 |
| Calcium-D-(+)-pantothenate | 0.1 |
| Vitamin B12 | 0.002 |
| p-Aminobenzoic acid | 0.1 |
| Thioctic acid | 0.1 |

### Example 2: Manufacture of Concentrated Clarified Broth

Fermentation was conducted similarly to Example 1. Biomass was filtered out. The clarified fermentation broth was concentrated up by water evaporation to reach total solids concentration of 52.6% by weight to form the clarified broth. Its density was 1.076 Kg/L. The formed clarified broth was analyzed. The results are summarized in Table 4.

TABLE 4
Example composition of clarified broth

| Component | Value | Units |
|---|---|---|
| Total solids | 52.6 | wt % |
| Density | 1.076 | kg/L |
| Ammonium Butyrate | 361.0 | g/kg |
| Ammonium Acetate | 112.0 | g/kg |
| Crude Protein (on AA basis) | 35.5 | g/kg |
| Crude Fat | 4.0 | g/kg |
| Ash | 5.5 | g/kg |
| Calcium | 0.1 | g/kg |
| Phosphorous | 0.4 | g/kg |
| Magnesium | 0.4 | g/kg |
| Potassium | 2.0 | g/kg |
| Sodium | 0.2 | g/kg |
| Iron | 6.0 | Ppm |
| Zinc | 5.0 | Ppm |
| Copper | <1 | Ppm |
| Manganese | 20 | Ppm |
| Molybdenum | 0.7 | Ppm |
| Sulfur | 200 | Ppm |

Amino acids (hydrolyzed values)

| | | |
|---|---|---|
| Lysine | 1.13 | g/kg |
| Methionine | 1.59 | g/kg |
| Threonine | 0.52 | g/kg |
| Aspartic acid/asparagine | 2.75 | g/kg |
| Glutamic acid/glutamine | 5.85 | g/kg |
| Serine | 0.66 | g/kg |
| Histidine | 0.30 | g/kg |
| Glycine | 1.17 | g/kg |
| Arginine | 0.68 | g/kg |
| Alanine | 13.47 | g/kg |
| Tyrosine | 0.45 | g/kg |
| Valine | 4.04 | g/kg |
| Phenylalanine | 0.74 | g/kg |
| Isoleucine | 0.65 | g/kg |
| Leucine | 0.93 | g/kg |
| Proline | 0.55 | g/kg |

### Example 3: Corn-Mash Fermentation

A fermentation similar to Example 1 was conducted except that instead of glucose, corn mash was used as the feedstock. Corn mash was diluted and filtered to a concentration of 60-160 g/L assumed glucose, and an amylase enzyme was added during the fermentation. Only trace elements (Table 2) and vitamins (Table 3) were added to the fermentation. Other operating conditions were the same and a similar production profile was achieved with a similar product as Example 2.

### Example 4: Removal of Phosphorous Ions

A concentrated clarified broth was manufactured similarly to Example 3. Its main components are shown in Table 5. Note that total phosphorous content was about 1800 mg/Kg and one half of it was in phosphate form. The rest is apparently in phytate form.

TABLE 5
Composition of the clarified broth

| Component | Value | Units |
|---|---|---|
| Total solids | 56.8 | wt % |
| Density | 1.076 | kg/L |
| Ammonium Butyrate | 369 | g/kg |
| Ammonium Acetate | 161 | g/kg |
| Ammonium lactate | 44 | g/Kg |
| Ash | 21 | g/kg |
| Calcium | 0.2 | g/kg |
| Total phosphorous | 1.6 | g/kg |
| Phosphate phosphorous | 0.8 | g/Kg |
| Magnesium | 1.3 | g/kg |
| Potassium | 6.7 | g/kg |
| Sodium | 1.7 | g/kg |

Samples of the concentrated clarified broth were treated by pH adjustment with NaOH solution to reach pH 8 or 9. At pH 9, a strong ammonia smell was felt. To some of the samples a calcium acetate solution was added. Then, the samples were centrifugation and supernatant was analyzed. Clarified broth samples were of 50 gram. The concentration of calcium acetate in the added solutions was 20% wt. The results are summarized in Table 5.

TABLE 5
Results of phosphorous reduction

| | | Calcium | | | | | |
|---|---|---|---|---|---|---|---|
| | | acetate | | | | | |
| | | solution | | | | | |
| Experiment | | added | Phosphate | Total P | Butyrate | Acetate | Lactate |
| # | pH | (ml) | P (mg/Kg) | (mg/Kg) | (gr/Kg) | (gr/Kg) | (gr/Kg) |
| Before | | | 800 | 1600 | 369 | 161 | 44 |
| treatment | | | | | | | |
| 4.1 | 8 | | 320 | 820 | 309 | 130 | 47 |
| 4.2 | | 0.75 | 230 | 710 | 295 | 137 | 51 |
| 4.3 | 8 | 1.5 | 160 | 560 | 303 | 130 | 44 |
| 4.4 | 9 | | 10 | 510 | 310 | 131 | 43 |
| 4.5 | 9 | 1.12 | 10 | 530 | 278 | 121 | 37 |

There results demonstrate that phosphate phosphorous is very efficiently reduced in treating such concentrated clarified broth. Such reduction is required for the use of the product in road anti-icing.

1. A method for manufacturing a butyrate product, comprising
(i) providing a carbon-source-comprising fermentation feedstock;
(ii) fermenting said feedstock with a Clostridia class bacterium which natively produces butyric acid, while maintaining a pH of about 5 to 7, wherein ammonia is used for said maintaining pH and whereby a fermentation broth is formed comprising ammonium butyrate, biomass and optionally a fermentation byproduct;
(iii) separating said biomass from said fermentation broth to form separated biomass and a clarified fermentation broth, wherein said clarified fermentation broth comprises said ammonium butyrate and optionally said fermentation byproduct; and
(iv) adding to said clarified broth an additive selected from the group consisting of a mineral acid, a mineral base, a soluble calcium salt and combinations thereof, whereby said butyrate product is formed in said clarified broth.

2. The method of claim 1, wherein said additive comprises a mineral acid, and wherein said butyrate product formed in said clarified broth is butyric acid in free acid form, the method further comprising separating said butyric acid in free acid form from said clarified broth by a method selected from the group consisting of distilling and liquid-liquid extraction of said butyric acid.

3. The method of claim 1, wherein said additive comprises a mineral acid, and wherein butyric acid in free acid form and an ammonium salt of said mineral acid are formed in said clarified broth, the method further comprising providing an alkanol, reacting said butyric acid in free acid form with said alkanol to form a butyrate ester, and separating said butyrate ester from said clarified broth by a method selected from the group consisting of distilling and liquid-liquid extraction of said butyric ester.

4. The method of claim 1, wherein said additive comprises a mineral base, wherein ammonia is liberated in said clarified broth, wherein said butyrate product formed in said clarified broth is a butyric salt of said mineral base, said the method further comprising separating said butyrate product from said liberated ammonia by vaporizing said liberated ammonia.

5. The method of claim 1, wherein at least one selected from the group consisting of said clarified fermentation broth, said carbon source and said carbon-source containing fermentation feedstock further comprises a phosphorous anion selected from the group consisting of phosphate, phytate and combinations thereof and said additive comprises a soluble calcium salt, wherein a water-insoluble calcium salt of said phosphorous anion is formed, the method further comprising separating said butyrate product from said insoluble salt.

6. The method of claim 5, wherein said carbon-source and/or said carbon-source containing fermentation feedstock comprises phytate, the method further comprising contacting said carbon-source and/or said carbon-source containing fermentation feedstock with a phytase enzyme, whereby phytase is hydrolyzed to form phosphate, wherein said contacting with phytase enzyme is conducted prior to or simultaneously with said fermenting.

7. The method of claim 1, wherein said mineral acid is selected from the group consisting of sulfuric acid, phosphoric acid and nitric acid and combinations thereof.

8. The method of claim 1, wherein said mineral base is selected from the group consisting of oxides, hydroxides, carbonates and bicarbonates of sodium, potassium, calcium and magnesium and combinations thereof.

9. (canceled)

10. The method of claim 1, wherein said fermentation broth further comprises ammonium acetate.

11. The method of claim 1, wherein said butyrate product is a feed ingredient.

12. The method of claim 1, wherein said butyrate product is an anti-icing agent, wherein said additive comprises a potassium base and wherein butyrate product comprises potassium butyrate.

13. (canceled)

14. The method of claim 1, wherein said additive comprises a mineral base, and wherein a butyrate salt of said mineral base is formed in said clarified broth, the method further comprising separating said butyrate salt of said mineral base from said clarified broth by at least one method selected from the group consisting of salt crystallization and electrodialysis.

15. The method of claim 3, further comprising crystallizing said ammonium salt from said clarified broth.

16. The method of claim 3, wherein said ammonium salt is selected from the group consisting of ammonium sulfate, ammonium phosphate, ammonium nitrate and a combination thereof.

17. The method of claim 3, further comprising reacting said ammonium salt with a calcium base, whereby ammonia is liberated and a precipitate is formed and wherein said precipitate comprises calcium sulfate, calcium phosphate or a combination thereof.

18. (canceled)

19. The method of claim 4, further comprising reusing said liberated ammonia and/or said vaporized ammonia for pH control in said fermentation.

20. (canceled)

21. (canceled)

22. The method of claim 1, further comprising at least partially removing water from said fermentation broth and/or from said clarified fermentation broth.

23. An animal feed comprising selected nutrients, said butyrate feed ingredient of claim 11 or a product thereof and optionally biomass.

24. The animal feed of claim 23, further comprising a mineral micronutrient.

25. (canceled)

26. (canceled)

27. A method for treating an animal comprising feeding said animal with the feed of claim 23.

28. The method of claim 27, wherein said feed comprises butyrate at a concentration between 10 ppm and 20,000 ppm.

29. (canceled)

30. (canceled)

31. (canceled)
